(19) United States
(12) Patent Application Publication
(10) Pub. No.: US 2022/0192696 A1
(43) Pub. Date: Jun. 23, 2022

(54) ULTRASONIC WAVEGUIDE AND BLADE FOR ULTRASONIC SURGICAL INSTRUMENTS AND METHOD OF MANUFACTURING THE SAME

(71) Applicant: Covidien LP, Mansfield, MA (US)

(72) Inventors: Matthew S. Cowley, Frederick, CO (US); Michael J. Brown, Superior, CO (US); Christopher T. Tschudy, Arvada, CO (US)

(21) Appl. No.: 17/600,733

(22) PCT Filed: Apr. 9, 2020
(86) PCT No.: PCT/US2020/027371

§ 371 Date: Oct. 1, 2021

Related U.S. Application Data
(60) Provisional application No. 62/835,277, filed on Apr. 17, 2019.

(51) Int. Cl. A61B 17/32

(52) U.S. Cl. CPC A61B 17/320068; A61B 2017/00526; A61B 2017/320082

(57) ABSTRACT

A waveguide configured for use with an ultrasonic surgical instrument includes an elongated body having a first engagement member at a proximal end thereof. The first engagement member engages the elongated body with an ultrasonic transducer to enable transmission of ultrasonic energy from the ultrasonic transducer along the elongated body. The elongated body being formed from titanium or a titanium alloy. A blade is fixedly engaged to a distal end of the elongated body, and distally extends from the distal end of the elongated body in order to receive ultrasonic energy from the elongated body for treating tissue in contact with the blade. The blade being formed from an amorphous material.

### BACKGROUND

### Technical Field

The present disclosure relates to ultrasonic surgical instruments and, more particularly, to an ultrasonic waveguide and blade for ultrasonic surgical instruments and a method of manufacturing the same.

### Background of Related Art

Ultrasonic surgical instruments utilize ultrasonic energy, i.e., ultrasonic vibrations, to treat tissue. More specifically, ultrasonic surgical instruments utilize mechanical vibration energy transmitted at ultrasonic frequencies to coagulate, cauterize, fuse, seal, cut, desiccate, fulgurate, or otherwise treat tissue.

Typically, an ultrasonic surgical instrument is configured to transmit ultrasonic energy produced by a generator and transducer assembly along a waveguide to an end effector that is spaced-apart from the generator and transducer assembly. With respect to cordless ultrasonic instruments, for example, a portable power source, e.g., a battery, and the generator and transducer assembly are mounted on the handheld instrument itself, while the waveguide interconnects the generator and transducer assembly and the end effector. Wired ultrasonic instruments operate in similar fashion except that, rather than having the generator and power source mounted on the handheld instrument itself, the handheld instrument is configured to connect to a standalone power supply and/or generator via a wired connection.

### SUMMARY

As used herein, the term “distal” refers to the portion that is being described which is further from a user, while the term “proximal” refers to the portion that is being described which is closer to a user. Further, to the extent consistent any or all of the aspects detailed herein may be used in conjunction with any or all of the other aspects detailed herein.

Provided in accordance with aspects of the present disclosure is a waveguide configured for use with an ultrasonic surgical instrument. The waveguide includes an elongated body having a first engagement member at a proximal end thereof that engages the elongated body with an ultrasonic transducer of an ultrasonic surgical instrument to enable transmission of ultrasonic energy from the ultrasonic transducer along the elongated body. The elongated body is formed from titanium or a titanium alloy. A blade is fixedly engaged to a distal end of the elongated body and extends distally therefrom to receive ultrasonic energy from the elongated body for treating tissue in contact with the blade. The blade is formed from an amorphous material.

In an aspect of the present disclosure, the elongated body defines a second engagement member at a distal end thereof, while the second engagement member facilitates the fixed engagement of the blade with the elongated body at the distal end of the elongated body.

In another aspect of the present disclosure, the blade is injection molded about the second engagement member to establish an interference fit bonding between the elongated body and the blade.

In still another aspect of the present disclosure, the second engagement member is a non-uniformly shaped protrusion that facilitates an interference fit bonding between the elongated body and the blade.

In yet another aspect of the present disclosure, the transmission of ultrasonic energy from the ultrasonic transducer to the waveguide generates a standing wave, having at least one anti-nodal point, between the proximal end and the distal end of the waveguide.

In still yet another aspect of the present disclosure, the blade is formed from a metallic amorphous material.

In another aspect of the present disclosure, the blade is formed from a metallic glass amorphous material.

A method of manufacturing a waveguide of an ultrasonic surgical instrument provided in accordance with aspects of the present disclosure includes forming an elongated body defining a non-uniformly shaped protrusion extending from a distal end of the elongated body and injection molding an amorphous material over the non-uniformly shaped protrusion to form a blade fixedly engaged with and extending distally from the elongated body.

In an aspect of the present disclosure, the injection molding forms an interference fit bonding between the elongated body and the blade.

In another aspect of the present disclosure, the interference fit bonding between the elongated body and the blade facilitates transmission of ultrasonic energy from an ultrasonic transducer to the waveguide such that a standing wave, having at least one anti-nodal point, is generated between the proximal end and the distal end of the waveguide.

In still another aspect of the present disclosure, the interference fit bond is positioned at an anti-nodal point of the standing wave along the waveguide.

In yet another aspect of the present disclosure, the amorphous material is metallic.

In still yet another aspect of the present disclosure, the amorphous material is a metallic glass.

In another aspect of the present disclosure, forming the elongated body includes machining the elongated body from a cylindrical rod.

In yet another aspect of the present disclosure, the elongated body is formed from titanium or a titanium alloy.

Also provided in accordance with aspects of the present disclosure is an ultrasonic surgical instrument. The ultrasonic surgical instrument includes a housing supporting an ultrasonic transducer, and an elongated assembly extending distally from the housing. The elongated assembly includes a waveguide including an elongated body having a first engagement member at a proximal end thereof. The first engagement member engages the elongated body with the ultrasonic transducer to enable transmission of ultrasonic energy from the ultrasonic transducer along the elongated body. The elongated body is formed from titanium or a titanium alloy. The waveguide further includes a blade fixedly engaged to and extending distally from a distal end of the elongated body to receive ultrasonic energy from the elongated body for treating tissue in contact with the blade. The blade is formed from an amorphous material. The ultrasonic surgical instrument further includes a fixed sleeve and a movable sleeve each disposed about the waveguide and defining a proximal end portion and a distal end portion. A jaw member is pivotably supported at the distal end portion of the fixed sleeve and operably coupled to the movable sleeve such that translation of the movable sleeve relative to the fixed sleeve pivots the jaw member relative to the blade between an open position and a clamping position.

In an aspect of the present disclosure, the elongated body defines a second engagement member at a distal end thereof. The second engagement member facilitates the fixed engagement of the blade with the elongated body at the distal end of the elongated body.

In another aspect of the present disclosure, the blade is injection molded about the second engagement member to establish an interference fit bonding between the elongated body and the blade.

In still another aspect of the present disclosure, the second engagement member is a non-uniformly shaped protrusion facilitating an interference fit bonding between the elongated body and the blade.

In yet another aspect of the present disclosure, the transmission of ultrasonic energy from the ultrasonic transducer to the waveguide generates a standing wave, having at least one anti-nodal point, between the proximal end and the distal end of the waveguide.

In yet still another aspect of the present disclosure, the blade is formed from a metallic amorphous material.

In another aspect of the present disclosure, the blade is formed from a metallic glass amorphous material.

### BRIEF DESCRIPTION OF THE DRAWINGS

The above and other aspects and features of the present disclosure will become more apparent in view of the following detailed description when taken in conjunction with the accompanying drawings wherein like reference numerals identify similar or identical elements.

FIG. 1 is a perspective view of an ultrasonic surgical instrument provided in accordance with the present disclosure;

FIG. 2 is a perspective view of the ultrasonic surgical instrument of FIG. 1 with the elongated assembly separated from the handle assembly;

FIG. 3 is an exploded, perspective view of the elongated assembly of FIG. 2;

FIG. 4 is an enlarged, longitudinal, cross-sectional view of a portion of the ultrasonic surgical instrument of FIG. 1 illustrating engagement between the elongated assembly and the handle assembly;

FIG. 5 is a perspective view of a waveguide in accordance with the present disclosure configured for use with the ultrasonic surgical instrument of FIG. 1; and

FIG. 6 is an enlarged, exploded view of a portion of the waveguide of FIG. 5 illustrating interference fit bonding between the elongated body and the blade of the waveguide.

### DETAILED DESCRIPTION

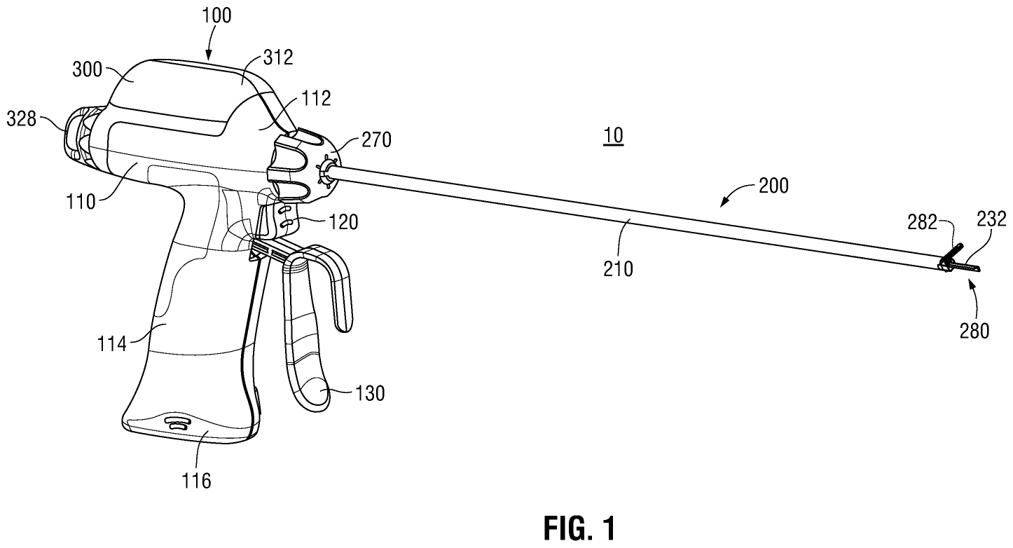

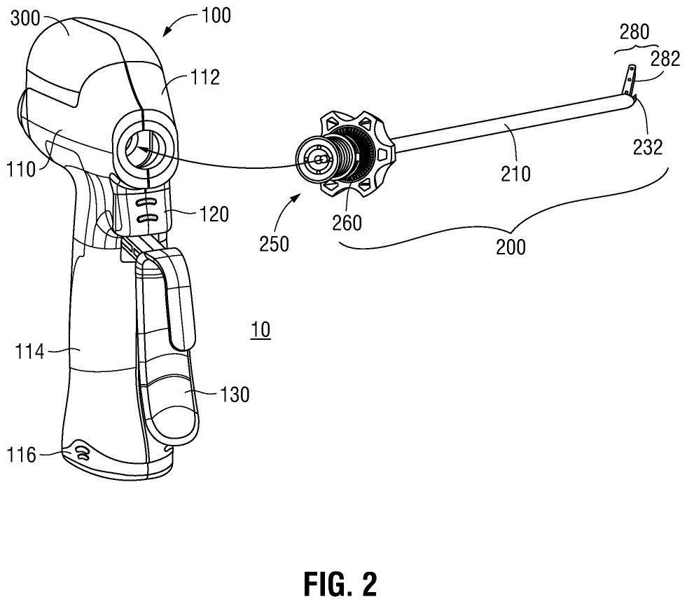

Referring generally to FIGS. 1 and 2, an ultrasonic surgical instrument provided in accordance with the aspects and features of the present disclosure is shown generally identified by reference numeral 10. Although detailed with respect to ultrasonic surgical instrument 10, the aspects and features of the present disclosure are equally applicable for use with any suitable ultrasonic surgical instrument. Thus, ultrasonic surgical instrument 10 is generally described hereinbelow. Additional features of ultrasonic surgical instrument 10, including the assembly and use thereof, are detailed in U.S. patent application Ser. No. 15/496,241, filed on Apr. 25, 2017 and published as Patent Application Publication No. US 2017/0319229, the entire contents of which are hereby incorporated herein by reference.

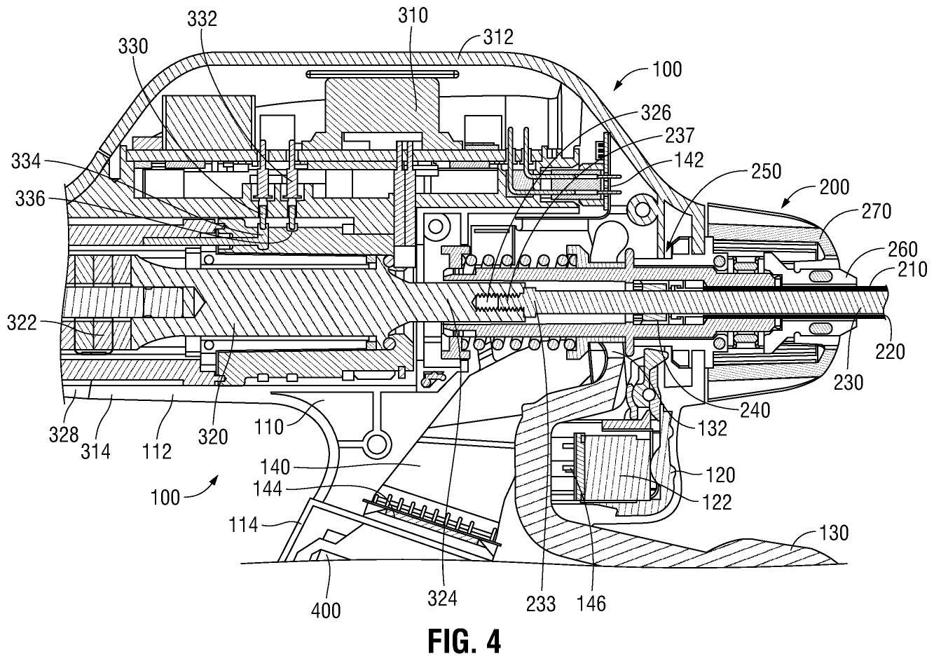

Ultrasonic surgical instrument 10 generally includes a handle assembly 100 and an elongated assembly 200 that is configured to releasably engage handle assembly 100. Handle assembly 100 includes a housing 110 defining a body portion 112 configured to support an ultrasonic transducer and generator assembly (“TAG”) 300, and a fixed handle portion 114 defining a compartment 116 configured to receive a battery assembly 400 (FIG. 4). Handle assembly 100 further includes an activation button 120 operably positioned to electrically couple between TAG 300 and battery assembly 400 (FIG. 4) when TAG 300 is mounted on body portion 112 of housing 110 and battery assembly 400 (FIG. 4) is engaged within compartment 116 of housing 110.

A clamp trigger 130 extends from housing 110 of handle assembly 100 adjacent fixed handle portion 114 of housing 110. Clamp trigger 130 includes a bifurcated drive portion 132 extending into body portion 112 of housing 110 and is selectively movable relative to housing 110 to actuate ultrasonic surgical instrument 10.

TAG 300 and battery assembly 400 (FIG. 4), as noted above, are each removable from handle assembly 100 to facilitate disposal of handle assembly 100 after a single use or to enable sterilization of handle assembly 100 for subsequent use. TAG 300 may be configured to withstand sterilization such that TAG 300 may be sterilized for repeated use. Battery assembly 400 (FIG. 4), on the other hand, is configured to be aseptically transferred and retained within compartment 116 of fixed handle portion 114 of housing 110 of handle assembly 100 such that battery assembly 400 (FIG. 4) may be repeatedly used without requiring sterilization thereof.

With additional reference to FIG. 4, an electrical connector 140 disposed within housing 110 of handle assembly 100 includes TAG contacts 142, battery assembly contacts 144, and an activation button connector 146. Electrical connector 140 electrically couples to activation button 120 via activation button connector 146,is configured to electrically couple to TAG 300 via TAG contacts 142 upon engagement of TAG 300 with body portion 112 of housing 110 of handle assembly 100, and is configured to electrically couple to battery assembly 400 via battery assembly contacts 144 upon engagement of battery assembly 400 within compartment 116 of fixed handle portion 114 of housing 110 of handle assembly 100. As such, in use, when activation button 120 is activated in an appropriate manner, an underlying two-mode switch assembly 122 is activated to supply power from battery assembly 400 to TAG 300 in either a “LOW” power mode or a “HIGH” power mode, depending upon the manner of activation of activation button 120.

Continuing with reference to FIGS. 1, 2, and 4, TAG 300 includes a generator 310 and an ultrasonic transducer 320. Generator 310 includes a housing 312 configured to house the internal electronics of generator 310, and a cradle 314 configured to rotatably support ultrasonic transducer 320. Ultrasonic transducer 320 includes a piezoelectric stack 322 and a distally-extending horn 324. Horn 324 defines a threaded female receiver 326 at the free distal end thereof. A set of connectors 330, 332 and corresponding rotational contacts 334, 336 associated with generator 310 and ultrasonic transducer 320, respectively, enable drive signals to be communicated from generator 310 to piezoelectric sack 322 to drive ultrasonic transducer 320. More specifically, piezoelectric stack 322 of ultrasonic transducer 320 converts a high voltage AC signal received from generator 310 into mechanical motion that is output from horn 324 to elongated assembly 200, as detailed below. Ultrasonic transducer 320 further includes a rotation knob 328 disposed at a proximal end thereof to enable rotation of ultrasonic transducer 320 relative to generator 310.

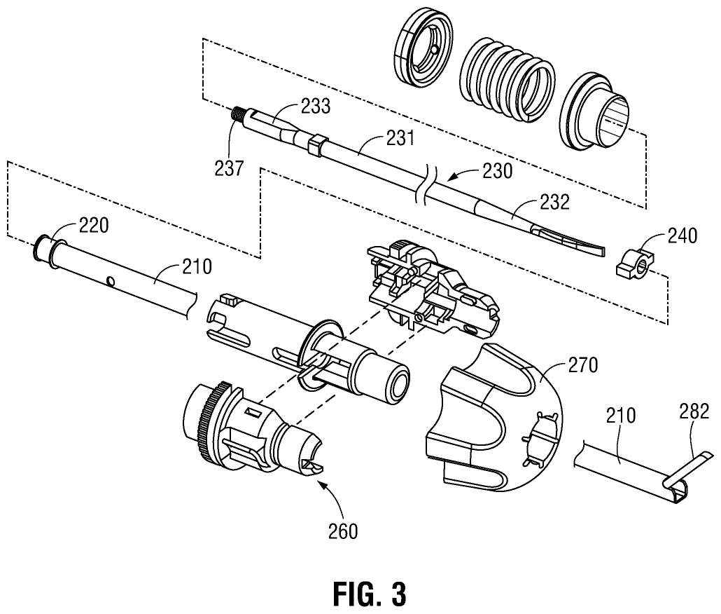

Referring to FIGS. 2-3, elongated assembly 200 includes an outer drive sleeve 210, an inner support sleeve 220 disposed within outer drive sleeve 210 and about which outer drive sleeve 210 is configured to slide, a waveguide 230 extending through inner support sleeve 220, a torque adapter 240 engaged about waveguide 230, a drive assembly 250 disposed about outer drive sleeve 210 and operably coupled between outer drive sleeve 210 and bifurcated drive portion 132 of clamp trigger 130 (FIG. 4), a torque housing 260 disposed about outer drive sleeve 210 and operably coupled to waveguide 230, a rotation knob 270 operably disposed about torque housing 260, and an end effector 280 (including a jaw member 282) disposed at the distal end of inner support sleeve 220. Elongated assembly 200 is configured to releasably engage handle assembly 100 such that mechanical motion output from horn 324 of ultrasonic transducer 320 is transmitted along waveguide 230 to end effector 280 for treating tissue therewith, such that clamp trigger 130 is selectively actuatable to manipulate end effector 280, and such that rotation knob 270 is selectively rotatable to rotate elongated assembly 200 relative to handle assembly 100. Elongated assembly 200 may be configured as a disposable, single-use component or a reusable component that is sterilizable for subsequent use. In embodiments, elongated assembly 200 is integrated with handle assembly 100 and, in such embodiments, is not removable therefrom.

With additional reference to FIGS. 4-6, waveguide 230, as noted above, extends through inner support sleeve 220. Waveguide 230 defines a body 231, a blade 232 extending from the distal end of body 231, and a first engagement member 233 extending from the proximal end of body 231. Blade 232 extends distally from inner support sleeve 220 and forms part of end effector 280 in that blade 232 is positioned to oppose jaw member 282 such that pivoting of jaw member 282 from the open position to the clamping position enables clamping of tissue between jaw member 282 and blade 232. Blade 232 defines a curved configuration wherein the directions of movement of jaw member 282 between the open and clamping positions are perpendicular to the direction of curvature of blade 232. However, it is also contemplated that blade 232 define a straight configuration or that blade 232 curve towards or away from jaw member 282, that is, where the directions of movement of jaw member 282 between the open and clamping positions are coaxial or parallel to the direction of curvature of blade 232.

First engagement member 233 of waveguide 230 is configured to enable engagement of waveguide 230 with horn 324 of ultrasonic transducer 320 such that mechanical motion produced by ultrasonic transducer 320 is capable of being transmitted along waveguide 230 to blade 232 for treating tissue clamping between blade 232 and jaw member 282 or positioned adjacent to blade 232. To this end, first engagement member 233 includes a threaded male shaft 237 that is configured for threaded engagement within threaded female receiver 326 of horn 324 of ultrasonic transducer 320. In other embodiments the first engagement member 233 includes a threaded female shaft configured to receive a threaded male shaft from horn 324. Any combination of mechanical couplings that allows for the ultrasonic waveform to be transmitted between the waveguide and horn will allow the device to function properly.

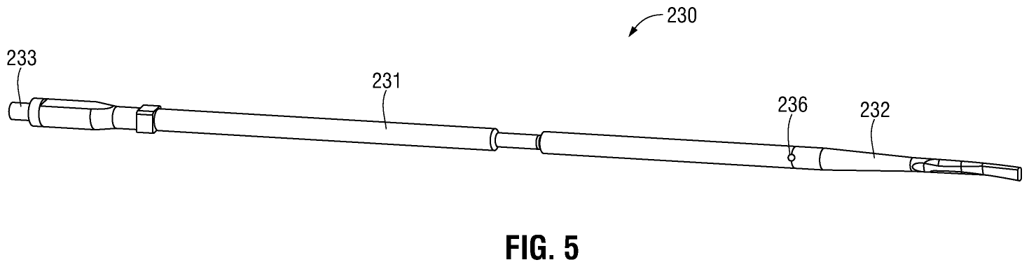

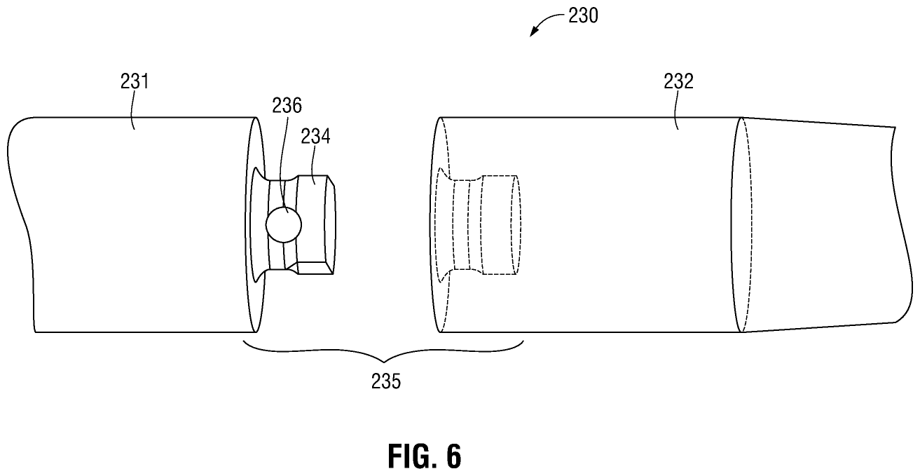

Referring to FIGS. 5 and 6, as noted above, blade 232 is fixedly engaged to a distal end of elongated body 231. Blade 232 extends distally from the distal end of elongated body 231 and is configured to receive ultrasonic energy from elongated body 231 for treating tissue in contact with blade 232, e.g., clamped between blade 232 and jaw member 282 (FIG. 1). In some embodiments, elongated body 231 is formed from titanium or a titanium alloy, and blade 232 is formed from an amorphous material.

Elongated body 231 of waveguide 230 defines a second engagement member 234 at a distal end thereof, such that second engagement member 234 facilitates the fixed engagement of blade 232 with elongated body 231 at the distal end of elongated body 231. Second engagement member 234 is configured to facilitate an interference fit bonding 235 between elongated body 231 and blade 232. Furthermore, second engagement member 234 is positioned to lie on an anti-nodal point 236 of a standing wave generated along waveguide 230 by the transmission of ultrasonic energy from the ultrasonic transducer 320. By positioning second engagement member 234 on or as close as possible to an anti-nodal point, the stress generated at this point, the point of engagement between elongated body 231 and blade 232, are at a minimum (while displacement is at a maximum).

In some embodiments, second engagement member 234 is a non-uniformly shaped protrusion configured to facilitate interference fit bonding 235 between elongated body 231 and blade 232. In other embodiments the second engagement member 234 is disposed on the the distal end of blade 232 rather than on the proximal end of the elongated body 231, such that the non-uniformly shaped protrusion is still configured to facilitate interference fit bonding 235 between elongated body 231 and blade 232. Additionally, in some embodiments, blade 232 is injection molded about second engagement member 234 to solidify and define interference fit bonding 235 between elongated body 231 and blade 232. The injection molding process allows for blade 232 to be formed from amorphous materials, e.g., metallic amorphous materials or metallic glass amorphous materials, that have higher material strength properties than the titanium or titanium alloys that are used to form elongated body 231. The injection molding process also avoids the added manufacturing cost of machining intricate features onto blade 232.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. A waveguide configured for use with an ultrasonic surgical instrument, the waveguide comprising:
an elongated body having a first engagement member at a proximal end thereof, the first engagement member configured to engage the elongated body with an ultrasonic transducer of an ultrasonic surgical instrument to enable transmission of ultrasonic energy from the ultrasonic transducer along the elongated body, wherein the elongated body is formed from titanium or a titanium alloy; and
a blade fixedly engaged to and extending distally from a distal end of the elongated body and configured to receive ultrasonic energy from the elongated body for treating tissue in contact with the blade, wherein the blade is formed from an amorphous material.

2. The waveguide according to claim 1, wherein the elongated body defines a second engagement member at a distal end thereof, the second engagement member facilitating the fixed engagement of the blade with the elongated body at the distal end of the elongated body.

3. The waveguide according to claim 2, wherein the blade is injection molded about the second engagement member to establish an interference fit bonding between the elongated body and the blade.

4. The waveguide according to claim 2, wherein the second engagement member is a non-uniformly shaped protrusion configured to facilitate an interference fit bonding between the elongated body and the blade.

5. The waveguide according to claim 1, wherein the transmission of ultrasonic energy from the ultrasonic transducer to the waveguide generates a standing wave, having at least one anti-nodal point, between the proximal end and the distal end of the waveguide.

6. The waveguide according to claim 1, wherein the blade is formed from a metallic amorphous material.

7. The waveguide according to claim 1, wherein the blade is formed from a metallic glass amorphous material.

8. A method of manufacturing a waveguide of an ultrasonic surgical instrument, the method comprising:
forming an elongated body defining a non-uniformly shaped protrusion extending from a distal end of the elongated body; and
injection molding an amorphous material over the non-uniformly shaped protrusion to form a blade fixedly engaged with and extending distally from the elongated body.

9. The method of manufacturing a waveguide according to claim 8, wherein the injection molding forms an interference fit bonding between the elongated body and the blade.

10. The method of manufacturing a waveguide according to claim 9, wherein the interference fit bonding between the elongated body and the blade facilitates transmission of ultrasonic energy from an ultrasonic transducer to the waveguide.

11. The method of manufacturing a waveguide according to claim 10, wherein the interference fit bond is positioned at an anti-nodal point along the waveguide.

12. The method of manufacturing a waveguide according to claim 8, wherein the amorphous material is metallic.

13. The method of manufacturing a waveguide according to claim 8, wherein the amorphous material is a metallic glass.

14. The method of manufacturing a waveguide according to claim 8, wherein forming the elongated body includes machining the elongated body from a cylindrical rod.

15. The method of manufacturing a waveguide according to claim 8, wherein the elongated body is formed from titanium or a titanium alloy.

16. An ultrasonic surgical instrument, comprising:
a housing supporting an ultrasonic transducer; and
an elongated assembly extending distally from the housing, the elongated assembly including:
a waveguide comprising:
an elongated body having a first engagement member at a proximal end thereof, the first engagement member configured to engage the elongated body with the ultrasonic transducer to enable transmission of ultrasonic energy from the ultrasonic transducer along the elongated body, wherein the elongated body is formed from titanium or a titanium alloy; and
a blade fixedly engaged to and extending distally from a distal end of the elongated body and configured to receive ultrasonic energy from the elongated body for treating tissue in contact with the blade, wherein the blade is formed from an amorphous material;
a fixed sleeve and a movable sleeve each disposed about the waveguide and defining a proximal end portion and a distal end portion; and
a jaw member pivotably supported adjacent the distal end portion of the fixed sleeve and operably coupled to the movable sleeve such that translation of the movable sleeve relative to the fixed sleeve pivots the jaw members relative to the blade between an open position and a clamping position.

17. The ultrasonic surgical instrument according to claim 16, wherein the elongated body defines a second engagement member at a distal end thereof, the second engagement member facilitating the fixed engagement of the blade with the elongated body at the distal end of the elongated body.

18. The ultrasonic surgical instrument according to claim 17, wherein the blade is injection molded about the second engagement member to establish an interference fit bonding between the elongated body and the blade.

19. The ultrasonic surgical instrument according to claim 17, wherein the second engagement member is a non-uniformly shaped protrusion configured to facilitate an interference fit bonding between the elongated body and the blade.

20. The ultrasonic surgical instrument according to claim 16, wherein the elongated body is further configured to be separable from a handle portion in the surgical instrument, and to be disposable after each use.
